(19) United States
(12) Patent Application Publication
(10) Pub. No.: US 2003/0170244 A1
(43) Pub. Date: Sep. 11, 2003

(54) Inhibition of Fas signaling

(76) Inventors: John D. Pluenneke, Parkville, MO (US); Timothy Connor, Madison, WI (US)

Correspondence Address: IMMUNEX CORPORATION, LAW DEPARTMENT, 51 UNIVERSITY STREET, SEATTLE, WA, 98101

(21) Appl. No.: 10/318,772

(22) Filed: Dec. 12, 2002

Related U.S. Application Data
(60) Provisional application No. 60/343,364, filed on Dec. 21, 2001.

(51) Int. Cl. A61K 39/395; A61K 38/23; A61K 38/18

(52) U.S. Cl. 424/146.1; 514/12

(57) ABSTRACT

The present invention relates to the general field of treating bone marrow failure and cancer. The invention, in part, utilizes inhibitors of Fas antigen (CD95) induced apoptosis to treat bone marrow failure and to improve cancer therapies.

This application claims the benefit of U.S. Provisional Application No. 60/343,364, filed Dec. 21, 2001, which is hereby incorporated by reference

### INTRODUCTION

The present invention relates to the general field of treating bone marrow failure and cancer. The invention, in part, utilizes inhibitors of Fas antigen (CD95) induced apoptosis to treat bone marrow failure and to improve cancer therapies.

### BACKGROUND

Programmed cell death, known as apoptosis is a cellular process that eliminates unneeded cells or cells that are potentially detrimental to a multi-cellular organism. In contrast to cell necrosis, apoptotic cellular elimination occurs in ordered steps starting with induction of condensation of the cytoplasm, followed by convolution of the plasma membrane, nuclear condensation, and ultimately by DNA fragmentation. Apoptosis can be initiated by an external signal, such as by serum withdrawal or DNA damage, but can also be initiated by cellular receptors. One such receptor, Fas, is a member of the nerve growth factor/tumor necrosis factor receptor superfamily and was identified and characterized by two separate agonist antibodies (CH11 and APO-1) to a cell surface antigen on a T cell line (Watanabe-Fukunaga et al., 1992, J. Immunol. 148:1274-79; Itoh et al., 1991, Cell 66:233-43). Both antibodies were shown to bind to Fas and induce apoptosis in the various different cell lines that express Fas (e.g., Yonehara et al., 1989, J. Exp. Med. 169:1748).

Fas mediated apoptosis has been shown to be involved in maintaining the proper balance of immune cells capable of reacting with and removing foreign antigens while preserving the integrity of self-recognition (Krammer, 2000, Nature 407:789-795). Fas has also been implicated in regulating hematopoiesis of various cell types and in the inappropriate deletion of erythrocytes, lymphocytes and/or myeloid cells (Bryder et al., 2001, J. Exp. Med. 194:941-952).

Fas additionally plays a role in the CD4-positive T cell killing of target cells (Shresta et al., 1998, Curr. Opin. Immunol. 10:581-7). Furthermore, some cancer cells expressing FasL have been shown to be resistant to T infiltrating lymphocytes (TIL) killing. This resistance, thought to be mediated through induction of apoptosis of attacking cells has been termed ‘tumor counterattack’ (Igney et al., 2000, Eur. J. Immunol. 30:725). Fas mediated apoptosis of TILs has been shown to contribute to tumor resistance to clearance in naturally occurring tumors (O'Connell et al., 2001, Nat. Med. 7(3):271-274). Other work speculates that additional factors (e.g., tumor growth factor beta (TGF-beta)) work in concert with Fas and to help ward off an anti-tumor immune response in some model systems (Chen et al., 1998, Science 282:1714-1717).

Thus, the present invention provides methods of manipulating FAS to promote the survival of desired hematopoietic cells in various disease states including anemia and cancer.

### SUMMARY

The present invention is directed to the treatment of bone marrow failure and cancer by the use of inhibitors of Fas mediated apoptosis. ‘Fas’ is also referred to as Fas antigen, Fas protein, Fas polypeptide, CD95, or APO-1.

In one aspect, the invention contemplates the treatment of bone marrow failure comprising administering to a patient in need thereof an effective amount of an inhibitor of Fas mediated apoptosis. Examples of bone marrow failures include aplastic anemia or myelodysplastic syndrome.

In another aspect, the invention contemplates a method of cancer therapy comprising co-administering an effective amount of a Fas inhibitor to a patient in need thereof in combination with an anti-cancer immune cell therapy. The immune cell therapy can comprise the administration to the patient of any one or more cell type selected from the group consisting of antigen primed dendritic cells, lymphocyte activated killer (LAK) cells, and tumor infiltrating lymphocytes (TIL). Immune cell therapies can also include the administration of immune cell activators such as flt3-ligand, agonist binding proteins of CD40 including antibodies and CD40L or fragments of CD40L, 4-1BB-L, agonist antibodies to 4-1BB, 4-1BB-L, interferon alpha, RANKL, a CD30 ligand antagonist, GM-CSF, TNF-α, IL-3, 1L-4, c-kit-ligand, and/or GM-CSF/IL-3 fusion proteins and combinations thereof.

### DETAILED DESCRIPTION

The present invention relates to the use of inhibitors of Fas mediated signaling to prevent inappropriate elimination of desired hematopoietic cells. For example, inhibitors of Fas signaling can be used to treat bone marrow failures, such as aplastic anemia and myelodysplastic syndromes by preventing elimination of erythroid cells or their progenitors. Further, Fas signaling inhibitors could be used to inhibit tumor induced apoptosis of anti-cancer cells administered as part of an immune cell therapy.

As used herein, the phrases “Fas mediated apoptosis”, “Fas signaling” or “Fas mediated cell death” refer to signaling by Fas which induces apoptotic cell death. One of skill in the art will recognize that these phrases are interchangeable. Fas mediated apoptosis is understood to mean signaling via Fas through various cytoplasmic effector molecules, namely, the “death inducing signaling complex” (DISC), resulting in programmed death of a cell. Programmed cell death is understood to mean the steps typically involved in apoptotic cell death such as membrane blebbing and fragmentation of DNA.

As used herein, the terms “inhibitor” or “antagonist” are meant to include various classes of molecules that are capable of interfering with a specified biological interaction and/or activity. Fas inhibitors or antagonists can include agents that target either Fas, FasL and/or downstream signaling molecules of Fas. These include, but are not limited to antibodies, soluble forms of a target polypeptide (also in multimer form), antisense nucleic acids, ribozymes, muteins, aptamers, and small molecules. Thus, the phrases “inhibition of Fas mediated apoptosis,” “inhibition of Fas signaling,” or “Fas inhibition” are all intended to indicate that under treatment conditions, there is a decreased capacity of Fas to transmit a signal relative to untreated conditions. In one example, inhibition of Fas signaling can be measured by a reduction in DNA fragmentation. In this example, inhibition can include minor reductions of DNA fragmentation, e.g., about 10% to 20%, or blockage, i.e., nearly 100% inhibition. One of skill in the art will readily appreciate that any standard apoptotic assay can be used to measure inhibition of Fas mediated apoptosis by an inhibitor.

It is to be understood that the term “treatment” is meant to encompass any reduction in the disease symptoms associated with the disease to be treated. Thus, for example, treatment of a patient with bone marrow failure would be demonstrated by increased red blood cell counts. Thus, as used herein, the term treatment includes amelioration of the disease up to and including a curative treatment, but is not intended to include only curative outcomes.

Treatment of Bone Marrow Failure With Fas Signaling Inhibitors

As used herein, the phrase “bone marrow failure” is defined as a disorder involving blood cells, typically erythroid (red blood cells), myeloid (white blood cells) and megakaryocytes (platelets), wherein mature cells are numerically deficient and/or malfunction relative to a healthy patient. For example, as used herein, bone marrow failure can be classified as a type of anemia due to the lack of red blood cells as a result of the failure of erythroid progenitor cells to proliferate and/or to differentiate. Also, secondary diseases can be the indirect result of bone marrow failure, such as when myeloid precursors fail, an infection can occur due to lack of immune protection.

Bone marrow failures can be inherited (i.e., genetic) or acquired through environmental exposure. Environmentally caused bone marrow failure can occur from exposure to any number of agents or conditions including but not limited to infectious agents such as viruses or bacteria, toxins, chemicals and/or natural diseases which result in abnormal control of the hematopoietic environment (Besa and Woermann, 2001, eMedicine J., volume 2(6)).

Specific non-limiting examples of bone marrow failure include aplastic anemia and myelodysplastic syndromes. Aplastic anemia is an often fatal disorder that occurs when the bone marrow stops producing enough of the three blood cells, i.e., red cells, white cells, and platelets. In these patients, their bone marrow is hypoplastic, namely containing very few blood forming cells. In myelodysplastic syndromes, the bone marrow largely stops making blood cells and those that are being produced are deformed or underdeveloped, which makes them function poorly. The bone marrow is usually described as hyperplastic, or stuffed with cells. A small percentage of myelodysplastic syndrome patients are hypoplastic making the disease look similar to aplastic anemia.

Other examples of bone marrow failures that can be treated by the methods of the invention include: anemia of chronic disease; aplastic anemia; including Fanconi's aplastic anemia; idiopathic thrombocytopenic purpura (ITP); myelodysplastic syndromes (including refractory anemia, refractory anemia with ringed sideroblasts, refractory anemia with excess blasts, refractory anemia with excess blasts in transformation); myelofibrosis/myeloid metaplasia; secondary thrombocytopenia in adults; acquired (autoimmune) hemolytic anemia; erythroblastopenia (RBC anemia); congenital (erythroid) hypoplastic anemia; and sickle cell vasocclusive crisis.

Current treatments for bone marrow failure rely primarily on administration of: 1) a factor or factors that stimulate a cell to secrete factors that promote hematopoiesis; 2) a growth factor or factors that can induce growth of the missing cell population; or 3) inhibitors that inhibit hematopoiesis repressors. Specific examples of treatments for bone marrow failure include TNF inhibitors, antithymocyte globulin, and/or immune cell activators such as GM-CSF, human granulocyte colony-stimulating factor (G-CSF), and/or erythropoietin, (e.g., sargramostim which is recombinant human granulocyte-macrophage colony stimulating factor (rhu GM-CSF), marketed as Leukine® and/or filgrastim which is G-CSF marketed as Neupogen® and/or erythropoietin, marketed as Epogen® (or erythropoietin with increased stability in the blood stream, darbepoetin alfa, which is marketed as Aranesp®).

The present invention provides a novel method of preventing the unwanted elimination of bone marrow cells by inhibiting a specific apoptotic signaling pathway, thereby providing a new method of treating bone marrow failures. Thus, in one embodiment, the invention provides a method for treating bone marrow failure comprising administering to a patient in need thereof an effective amount of an inhibitor of Fas mediated apoptosis. This therapy can be as a sole therapy or can be co-administered with an existing therapeutic agent as part of a combination therapy. Additionally, it is contemplated that the method of the invention used in combination with an existing anemia treatment can result in a synergistic reduction in disease symptoms. Thus, it will be understood that the methods of the invention can be used alone or in combination with current treatments, or alternatively with treatments yet to be developed for bone marrow failure.

Treatment of Cancers with Fas Signaling Inhibitors

Fas signaling inhibitors can be used to treat cancer or to augment cancer treatments by co-administration of a Fas signaling inhibitor with an anti-cancer immune cell therapy.

FasL can be expressed on the surface of tumor cells. When the immune system responds to the tumor cells and initiates a response, the FasL on the tumor cell can interact with Fas when it is expressed on a tumor infiltrating lymphocyte (TIL), inducing apoptosis of the attacking TILs. Accordingly, the tumor cells can kill the attacking lymphocytes before the lymphocytes can trigger the death of the cancer cells; a process known as ‘tumor counterattack.’ The present invention provides methods to protect an immune cell from being killed by tumor counterattack. Thus, in one embodiment, the invention provides a method comprising administering to a cancer patient in need thereof an effective amount of an inhibitor of Fas mediated apoptosis.

The present invention also provides methods to enhance immune cell therapies designed to stimulate an anti-cancer immune response comprising co-administering an inhibitor of Fas mediated apoptosis with anti-cancer immune cell therapies. As used herein, the phrase “anti-cancer immune cell therapies” refers to any therapy that utilizes immune cells to fight cancer. Examples of such therapies include the use of antigen presenting cells (e.g., dendritic cells) such as antigen primed dendritic cells where the dendritic cells are primed with a tumor antigen, lymphoid cells, (e.g., lymphocyte activated killer (LAK) cells and/or TILs) T cells cultured ex vivo with IL-2, and/or factors that stimulate the proliferation and/or activation anti-tumor cells (e.g., flt3-ligand, agonist binding proteins of CD40 including agonist antibodies to CD40 and CD40L or fragments of CD40L, 4-1BB-L, agonist antibodies to 4-1BB, 4-1BB-L, interferon alpha, RANKL, a CD30 ligand antagonist, GM-CSF, TNF-α, IL-3, IL-4, c-kit-ligand, and/or GM-CSF/IL-3 fusion proteins).

Cancers that can be treated using the methods of the invention include, but are not limited to, blood cell cancers such as autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemia, T gamma lymphoproliferative disease, cutaneous B cell lymphoma, cutaneous T cell lymphoma (i.e., mycosis fungoides), Sezary syndrome, acute myelogenous leukemia, chronic or acute lymphoblastic leukemia and hairy cell leukemia. Additional cancers that can be treated by the methods of the invention include, solid tumors, including sarcoma, osteosarcoma, and carcinoma, such as adenocarcinoma (for example, breast cancer) and squamous cell carcinoma Epstein-Barr virus-positive nasopharyngeal carcinoma, glioma, colon, stomach, prostate, renal cell, cervical and ovarian cancers, lung cancer (small cell lung carcinoma (SCLC) and non-small cell lung carcinoma (NSCLC)).

Malignancies with invasive metastatic potential can also be treated with the methods of the invention, including multiple myeloma. By treatment of the above described cancers, it is contemplated that symptoms associated with cancer will be relieved or ameliorated, such as cancer-associated cachexia, fatigue, asthenia, paraneoplastic syndrome of cachexia and hypercalcemia. One of skill in the art will recognize that immune cell therapies can be supplemented with yet additional therapeutic agents including anti-cancer drugs and/or anti-nausea drugs and/or any other drugs capable of benefiting the patient being treated.

In yet another embodiment, in cases where a patient is being treated for a solid tumor or a tumor that has metastasized, it is contemplated that the co-administration of the Fas inhibitor with an immune cell activator follows surgical reduction of the tumor mass. In addition, it is contemplated that the patient can be treated in an early stage in the disease progression so that that the patient is not immunologically suppressed or exhausted.

Inhibitors of Fas Mediated Apoptosis

There are a variety of non-limiting ways to inhibit Fas signaling. In one example, the inhibitor can disrupt transcription and/or translation of Fas or FasL messenger RNAs, for example, by expressing antisense Fas nucleic acids, inhibitory RNA or RNAi (Martinez et al., 2002, Cell, 110:563-574) or ribozymes. In another example, Fas can be prevented from binding to the FasL by targeting either Fas or the FasL with specific antibodies that bind to the ligand or receptor and prevent Fas/FasL interaction. In yet another example, Fas mediated apoptosis is inhibited by targeting of downstream molecules of Fas signaling. For example, DISC formation can be inhibited thereby blocking the apoptotic cascade, or additionally, caspase activity can be inhibited with specific inhibitors, e.g., caspase-8 inhibitors (Krammer, 2000, Nature 407:789-795). Additional specific examples of methods of inhibiting Fas that can be used in the present invention are discussed below.

Nucleic Acid Inhibitors

Fas expression can be inhibited to prevent Fas signaling, for example, by using antisense RNA or ribozyme approaches to inhibit or prevent translation of Fas and/or FasL, as described in the following section. It is to be understood that additional inhibitors based on nucleic acids can be used in the present methods including but not limited to inhibitor RNA (RNAi; Martinez et al, 2002, Cell, 110:563-574) and triple helixes (Rininsland et al., 1997, PNAS., 94:5854-9) as well as technologies yet to be discovered.

Antisense technology involves designing oligonucleotides (either DNA or RNA) that are complementary to Fas and/or FasL mRNA. The antisense oligonucleotides will bind to the complementary Fas and/or FasL mRNA transcripts and prevent translation. Absolute complementarity is not required. A sequence “complementary” to a portion of an RNA, as referred to herein, means a sequence having sufficient complementarity to be able to hybridize with the RNA to form a stable duplex. Oligonucleotides complementary to either the 5′- or 3′-non-translated, non-coding regions of the Fas and/or FasL mRNA can be used in an antisense approach to inhibit translation of endogenous Fas and/or FasL mRNA. Antisense nucleic acids can be at least six nucleotides in length, and can be oligonucleotides ranging from 6 to about 50 nucleotides in length. In specific aspects the oligonucleotide is at least 10 nucleotides, at least 17 nucleotides, at least 25 nucleotides or at least 50 nucleotides.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO88/09810, published Dec. 15, 1988), or hybridization-triggered cleavage agents or intercalating agents. (See, e.g., Zon, 1988, Pharm. Res. 5:539-549). The antisense oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. USA 85:7448-7451).

The antisense molecules should be delivered to cells which express the Fas mediated apoptosis transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. One approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Vectors can be plasmid, viral, or others known in the art, used for replication and expression in mammalian cells.

Ribozymes designed to catalytically cleave Fas and/or FasL mRNA transcripts can also be used to prevent translation and expression of Fas and/or FasL protein. (See, e.g., PCT International Publication WO 90/11364; U.S. Pat. No. 5,824,519). The ribozymes that can be used in the present invention include hammerhead ribozymes (Haseloff and Gerlach, 1988, Nature 334:585-591), RNA endoribonucleases (hereinafter “Cech-type ribozymes”) such as the one which occurs naturally in Tetrahymena thermophila (known as the IVS, or L-19 IVS RNA), described in PCT Publication No. WO 88/04300 and Been and Cech, 1986, Cell 47:207-216.

As in the antisense approach, the ribozymes can be composed of modified oligonucleotides (e.g. for improved stability, targeting, etc.) and should be delivered to cells which express the Fas and/or FasL polypeptide in vivo. One method of delivery involves using a DNA construct “encoding” the ribozyme under the control of a strong constitutive pol III or pol II promoter, so that transfected cells will produce sufficient quantities of the ribozyme to destroy endogenous Fas and/or FasL polypeptide messages and inhibit translation. Because ribozymes, unlike antisense molecules, are catalytic, a lower intracellular concentration is required for efficiency.

Protein-Based Fas Signaling Inhibitors

Protein-based therapeutics can also be used to inhibit the activity of Fas, such as antibodies specific for Fas or FasL polypeptides that inhibit the ligand-receptor interaction can be used to inhibit Fas activity. It is to be understood that additional non-antibody inhibitors can be used, such as for example, soluble extracellular domain portions of Fas or FasL and/or peptibodies can be used in the methods of the invention.

For the production of antibodies, various host animals can be immunized by injection with Fas or FasL polypeptides, functional equivalents and/or fusions thereof. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, and the like.

Monoclonal antibodies can be obtained by any technique which provides for the production of antibody molecules by continuous cell lines in culture. These include, but are not limited to, the hybridoma technique of Kohler and Milstein (U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo resulting in production of high titers of mAbs.

Working examples of antibodies capable of inhibiting Fas are provided in U.S. Pat. Nos. 5,620,889, 5,830,469, and 6,015,559, relevant portions of each of which are incorporated herein in their entirety.

A chimeric antibody is a molecule in which different portions are derived from different animal species, such as those having a variable region derived from a porcine antibody and a human immunoglobulin constant region (Takeda et al., 1985, Nature 314:452-454). Chimeric antibodies can be generated by splicing the portion of the cDNA that encodes the antibody recognition domain in the proper orientation onto a cDNA encoding the constant region of a human antibody. Because the majority of the chimeric antibody is of human origin, it has reduced immunogenicity relative to the non-human antibody.

For use in humans, the antibodies need not be, but are preferably human or humanized antibodies. Such human or humanized antibodies can be made by well known techniques and are commercially available from, for example, Medarex Inc. (Princeton, N.J.) and Abgenix Inc. (Fremont, Calif.). Human antibodies are understood to be antibodies that have sequences derived almost entirely from the human coding sequence thereby minimizing their immunogenicity. Humanized antibodies are understood to be non-human antibodies that have specific residues mutagenized to correspond to human antibodies to decrease immunogenicity in humans.

Antibody fragments can be used according to the invention, for example, F(ab′)₂ fragments, which can be produced by pepsin digestion of the antibody molecule or Fab fragments which can be generated by reducing the disulfide bridges of the (ab′)₂ fragments. Alternatively, Fab expression libraries can be used to identify monoclonal Fab fragments with the desired specificity (Huse et al., 1989, Science 246:1275-1281). Single chain antibodies can also be used according to the invention and are formed by linking the heavy and light chain fragments of the Fv region via an amino acid bridge, resulting in a single chain polypeptide (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546).

Soluble truncated fragments of Fas and/or FasL polypeptides can also be employed in inhibiting a biological activity of Fas. Inhibition occurs by binding to either the ligand or receptor and thereby blocking interaction of a corresponding binding partner that would activate Fas mediated apoptosis. Encompassed within the invention are soluble portions of the extracellular domain of Fas and/or FasL polypeptides that act as “dominant negative” inhibitors of Fas and/or FasL polypeptide function when expressed as fragments. For example, a purified polypeptide domain of Fas can be administered to a patient that would bind to the FasL in a non-functional manner and prevent binding of FasL to native Fas, thereby blocking signaling by the bound FasL.

The inhibitory Fas and/or FasL polypeptides can also be produced as fusion proteins to heterologous polypeptide sequences. It is contemplated that the heterologous sequence comprises a functional activity that would enhance the Fas inhibitory activity of molecule. For example, the heterologous sequence could be a Fc domain of an antibody, a leucine zipper domain, or any other known, or yet to be discovered, domain or epitope that facilitates structure or the purification of the inhibitory polypeptide upon recombinant expression. Additionally, it is contemplated that the heterologous sequences can selected based upon their ability to enhance inhibition of Fas activity, and/or to increase solubility of the fusion polypeptide, thereby easing purification and preparation in compositions for administration to patients.

Rational Design of Compounds that Inhibit Fas Signaling

The goal of rational drug design is to produce structural analogs of biologically active polypeptides of interest or of small molecules with which they interact, e.g., inhibitors, blocking molecules, and/or antagonists, etc. Any of these examples can be used to fashion drugs which are more active or stable forms of the polypeptide or which enhance or interfere with the function of a polypeptide in vivo (Hodgson J, 1991, Biotechnology 9:19-21).

In one approach, the three-dimensional structure of a polypeptide of interest, or of a Fas-inhibitor complex, is determined by x-ray crystallography, by nuclear magnetic resonance, or by computer homology modeling or, most typically, by a combination of these approaches (Weber and Vincenz, 2001, FEBS 492:171-6). Both the shape and charges of the polypeptide must be ascertained to elucidate the structure and to determine active site(s) of the molecule. Less often, useful information regarding the structure of a polypeptide can be gained by modeling based on the structure of homologous polypeptides (Weber and Vincenz, 2001, FEBS 492:171-6) as has been conducted with the Fas and FADD death domain complex. In both cases, relevant structural information is used to identify efficient inhibitors or to identify small molecules that bind Fas polypeptides. Useful examples of rational drug design include molecules which have improved activity or stability as shown by Braxton S and Wells J A (1992, Biochemistry 31:7796-7801) or which act as inhibitors or antagonists of peptides as shown by Athauda S B et al (1993, J Biochem 113:742-746).

The use of Fas and/or FasL structural information in molecular modeling software systems to assist in inhibitor design is also encompassed by the invention. A particular method of the invention comprises analyzing the three dimensional structure of Fas or FasL polypeptides for binding sites, synthesizing a new molecule that putatively binds Fas or FasL, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original antigen. The anti-id could then be used to identify and isolate peptides from banks of chemically or biologically produced peptides. The isolated peptides would then act as the pharmacore.

Immune Cell Therapies

Inhibitors of Fas can be used in combination with anti-cancer immune cell therapies that comprise administering immune cells primed to attack cancer cells, or stimulating endogenous immune cells to attack cancer cells. However, the immune cells can express Fas, and the cancer cells can express FasL. Thus, in the absence of a Fas signaling inhibitor, the anti-cancer immune cells can killed by the cancer cells in a reverse killing process known as tumor counterattack. According to the methods of the invention, the co-administration of a Fas signaling inhibitor with anti-cancer immune cell therapies would diminish the ability of the cancer cell to kill an attacking cell before it was elminated itself. Examples of such therapies include co-administering one or more Fas signaling inhibitors with antigen presenting cells such as, e.g., antigen primed dendritic cells, and/or lymphoid cells, such as, e.g., lymphocyte activated killer (LAK) cells, and tumor infiltrating lymphocytes (TIL). Alternatively, such therapies include co-administering one or more immune cell activators, such as an immune cell stimulating cytokine, as discussed below.

Antigen Presenting Cells

Antigen presenting cells (APCs) are a general classification of different cell types that are capable of internalizing, processing and presenting antigens to secondary immune cells. Within the APC group, there are differences in efficiency of presentation of antigens, and the most effective antigen presenting cells are often called “professional APCs.” The more notable members of this group includes dendritic cells.

As used herein, the term “dendritic cells” refers to dendritic precursor cells that have matured and now have a morphology that is characterized by membrane extensions (known as dendrites, pseudopods, or veils), that are often up to several hundred micrometers long. Additional morphologic features of dendritic cells include high concentrations of intracellular structures related to antigen processing such as endosomes, lysosomes, and the Birbeck granules of Langerhans cells (LC) of the epidermis. Mature dendritic cells can be activated to be antigen presenting cells that, after being pulsed with an antigen, can then activate naive CD8 positive cytotoxic T lymphocytes (CTL) to initiate a primary immune response. Dendritic cells are derived from dendritic precursor cells that do not have a dendritic morphology and are not competent to elicit a primary immune response as antigen presenting cells.

Dendritic cells are molecularly characterized by surface molecules, in particular, by high expression of class II MHC antigens, and by the absence of other lymphocyte lineage markers, for example CD3, which is characteristic of T cells. Also present on dendritic cells are various adhesion and costimulatory molecules. Examples of adhesion molecules include but are not limited to CD11a (LFA-1), CD11c, CD 35, CD50 (ICAM-2), CD54 (ICAM-1), CD58 (LFA-3), and/or CD102 (ICAM-3). Costimulatory molecules such as CD80 (B7.1) and CD86 (B7.2), and molecules regulating costimulation such as CD40 are also expressed on mature cells. Additional dendritic cell markers can include, but are not limited to CD1, CD4, CD86, DEC-205, CD40 and/or HLA-DR in any combination, and the lack CD14. This unique molecular distinction facilitates purification of dendritic cells and also simplifies their identification.

As noted above, dendritic cell molecular phenotypes vary with the stage of maturation and activation. Human dendritic cell precursors in the peripheral blood initially can express CD2, 4, 13, 16, 32, and 33, but they gradually lose their expression of these antigens with maturation. In contrast, expression of adhesion molecules, costimulatory molecules, and MHC antigens increase with maturation. Some dendritic cells express FcR (CD16, CD32) and complement receptors (CD11b, CD11c, and CD35). CD11c can additionally act as a receptor for LPS as dendritic cells lack CD14, the usual LPS receptor, yet respond to LPS stimulation. Ordinarily, CD86 is expressed early in maturation, while relative to CD86, CD80 expression is later. CD80 and 86 are both upregulated with activation, particularly with CD40 mediated activation.

Further, antibodies have been identified that recognize antigens expressed on mature dendritic cells, and as such, are helpful in characterizing dendritic cell isolates used in the methods of the present invention. One example is anti-CD83, which recognizes mature activated dendritic cells, but not precursors, and also cross-reacts with activated B cells. Another example is anti-CMRF-44 which recognizes peripheral blood and activated dendritic cells (see generally, Nestle, 2000, Oncogene, 19:6673).

Isolating Antigen Presenting Cells

Isolation of the hematopoietic stem or progenitor cells can be performed by using, for example, affinity chromatography, antibody-coated magnetic beads, or antibodies fixed to a solid matrix, such as glass beads, flasks, etc. Antibodies that recognize a stem or progenitor cell surface marker can be fused or conjugated to other chemical moieties such as biotin—which can be removed with an avidin or a streptavidin moiety secured to a solid support; fluorochromes useful in fluorescence activated cell sorting (FACS), or the like. Isolation can be accomplished by an immunoaffinity column. Immunoaffinity columns can take any form, but usually comprise a packed bed reactor. The packed bed in these bioreactors can be made of a porous material having a substantially uniform coating of a substrate. The porous material, which provides a high surface area-to-volume ratio, allows for the cell mixture to flow over a large contact area while not impeding the flow of cells out of the bed. Typical substrates include avidin and streptavidin, while other conventional substrates can be used. The substrate should, either by its own properties, or by the addition of a chemical moiety, display high-affinity for a moiety found on the cell-binding protein such as a monoclonal antibody.

The monoclonal antibodies recognize a cell surface antigen on the cells to be separated and are typically further modified to present a biotin moiety. It is well known that biotin has a high affinity for avidin, and the affinity of these substances thereby removably secures the monoclonal antibody to the surface of the packed bed. Such columns are well known in the art, see Berenson, et al., J. Cell Biochem. 10D:239, 1986. The column is washed with a PBS solution to remove unbound material. Target cells can be released from the beads using conventional methods. Immunoaffinity columns of the type described above that utilize biotinylated anti-CD34 monoclonal antibodies secured to an avidin-coated packed bed are described for example, in PCT International Publication WO 93/08268. A variation of this method utilizes cell binding proteins, such as the monoclonal antibodies as described above, removably-secured to a fixed surface in the isolating means. The bound cell binding protein then is contacted with the collected cell mixture and allowed to incubate for a period of time sufficient to permit isolation of the desired cells.

Alternatively, the monoclonal antibodies that recognize cell surface antigens can be labeled with a fluorescent label, e.g., chromophore or fluorophore, and separated by cell sorting according to the presence of absence or the amount of labeled product.

The collected cells are then exposed to factors such as flt3-ligand alone or flt3ligand in concurrent or sequential combination any of: an agonist binding protein of CD40 including antibodies to CD40 or CD40L or fragments of CD40L, 4-1BB-L, agonist antibodies to 4-1BB, 4-1BB-L, interferon alpha, RANKL, a CD30 ligand antagonist, GM-CSF, TNF-α, IL-3, IL-4, c-kit-ligand, and/or GM-CSF/IL-3 fusion proteins and combinations thereof. The precursor cells then are allowed to differentiate and commit to cells of the dendritic lineage. The dendritic cells are collected and can either be (a) administered to a patient in order to augment the immune system and T-cell mediated or B-cell mediated immune responses to antigen, (b) exposed to an antigen prior to administration of the dendritic cells into a patient, (c) transfected with a gene encoding an antigen-specific polypeptide or (d) exposed to an antigen and then allowed to process and present the antigen, ex vivo, to T-cells collected from the patient followed by administration of the antigen-specific T-cells to the patient.

Priming Antigen Presenting Cells

Prior to administration to a patient, dendritic cells can be pulsed with antigen in order to enhance presentation of specific antigen to an immune effector cell, such as a cytotoxic T cell lymphocyte (CTL). Typically this is done after purification of the dendritic cells, i.e., ex vivo, however, in cases where the dendritic cells are not purified, the antigen pulsing is with unpurified cells, e.g., in the subject.

Several methods can be used to pulse dendritic cells with antigen ex vivo to make them effective or competent to activate a desired subset of CTL. For example, antigen presenting cells such as dendritic cells can be exposed to unpurified whole cell lysates, (e.g., tumor cell lysates), to purified polypeptides or to purified antigenic peptides (e.g., tumor specific polypeptides), where these molecules are then processed by the cells for presentation to effector cells. When purified peptides are pulsed into antigen presenting cells, the peptides are processed through the “endogenous” class I pathway such that they are presented in association with MHC class I molecules, and accordingly are able to activate CD8 positive CTL.

In addition to peptides, certain polypeptides or proteins can be introduced to antigen presenting cells such that the polypeptides or proteins are processed through the MHC class I, as opposed to class II, pathway (see, for example, Mehta-Damani, A., et al., 1994, J. Immunol. 153:996). The incorporation of these polypeptide or protein antigens into liposomes has been used to move antigens into antigen presenting cells such as dendritic cells (e.g., Nair, S., et al., 1992, J. Immunol. Meth. 152:237).

Selected antigens can also be introduced to antigen presenting cells by transfection with expression vectors containing genes encoding such antigens. Transfection of antigen presenting cells with a gene encoding a desired antigen is an effective way to express the antigen in association with the class I MHC. Any of a variety of known methods (see, for example, Ausubel, F. M., et al., Current Protocols in Molecular Biology, John Wiley and Sons, Inc., Media Pa.; and Mulligan, R. C., 1993, Science 260:926) can be used for such transfections, including calcium phosphate precipitation, lipofection, naked DNA exposure, as well as viral vector-based approaches, such as retroviral, adenoviral, adeno-associated virus, and vaccinia virus vectors. Further methods of priming dendritic cells include exposing the dendritic cells to RNA from the target cell (Bordignon et al., 1999, Haematolgica, 84:1110-1149).

Another exemplary method describes inducing a specific anti-tumor cytotoxic T cell response in vitro and in vivo, wherein the therapeutic compositions consist of antigen presenting cells activated by contact with a polypeptide complex constructed by joining together a dendritic cell-binding protein and a polypeptide antigen (U.S. Pat. No. 6,080,409).

Immune Cell Therapy with Lymphocytes

In another embodiment, the invention contemplates co-administration of Fas inhibitors to a cancer patient in need thereof with an effective amount of an immune cell therapeutic selected from the group consisting of lymphocyte activated killer (LAK) cells and/or tumor infiltrating lymphocytes (TIL). In this embodiment, the lymphoid cells are harvested and grown ex vivo prior to co-administration into the cancer patient and represent an alternative embodiment for the immune cell therapies used in the methods of the present invention.

LAK cells

LAK cells were originally identified as lymphoid cells primarily found in the peripheral blood that were capable of lysing neoplastic cells in vitro in the presence of supraphysiological levels of IL-2, e.g., 500 to 1000 IU/ml (Hoffman et a., 2000, Seminars in Oncology, 27:221-233). These cells can be harvested from healthy donors and have been shown to be active in cancer patients with solid tumors. LAK cells are able to lyse target cells from syngeneic, allogeneic or xenogeneic sources are non-major histocompatibility class I restricted.

LAK cells can be obtained from either regional lymph nodes or peripheral blood. These LAK cells are then typically grown in media comprising IL-2 for a period of time resulting in expansion of the LAK cell population. In a specific non-limiting example, the LAK cells are grown in media comprising IL-2 (150 U/ml, Shionogi Company, Japan) for 2 to 3 weeks. The cells can then be stored in liquid nitrogen or any other suitable cryopreservation storage facility until use (Kimura and Yamaguchi, 1997, Cancer, 80:42-49). LAK cells are prepared for administration by standard methods and can typically be administered at a dose of 1-5×10⁹ cells/injection. The LAK cells can be administered with IL-2, or prior to, or after IL-2 administration.

TIL

TIL can be derived from solid tumors that have been resected or from tumor biopsies. These cells have much higher immunospecificity to tumor cells than LAK cells, at least in vitro, and thus can be administered at lower doses relative to LAK cells (Hoffman et a., 2000, Seminars in Oncology, 27:221-233).

TIL can be isolated, for example, as follows. Isolated tumor fragments are subdivided into small fragments, approximately 5 mm diameter, which are cultured separately in 24 well plates in media comprising RPMI 1600 with serum and recombinant IL-2 (30 Units (Chiron, Calif.)) and 15% conditioned media from PHA activated lymphocytes. Within four days, radial growth from the tumor cells should be visible, which then proliferate rapidly over the following week. TIL cells are pooled from the wells after a total culture time of two weeks, and cryopreserved until needed. Alternatively, the cells can be immediately put into use and not frozen.

Tumor cells can be isolated by taking a small amount of tumor tissue and enzymatically digesting it for about four hours, in, for example, RPMI 1640 media containing collegenase, deoxyribonuclease and hyalurinodase, followed by separation in density gradient centrifugation. The cells in the interface can then be cultured in RPMI 1640 media comprising serum and other immune cell activators. A rapidly growing cell line is then isolated from repeat passaging. Additionally, once isolated, the tumor cell line can be transfected with a construct encoding IL-2 and selected for secretion of this interleukin.

Newly thawed TIL can be grown in the media described above for three days and then exposed to the tumor cells described above by seeding plates with 70% tumor cells, followed by irradiation. The TIL should be recovered and plated on new tumor cells within 3-4 days as the tumor cells are typically severely damaged by the co-cultivation (Schendel et al., 1993, J. Immunol., 151:4209).

It was recently demonstrated that tumor specific killing could be improved by a further selection for interferon-gamma producing TIL (Becker et al., 2001, Nature Med., 7:1159). This technique involves selecting as described above, followed by selection of TIL that express interferon-gamma by stimulation of the TIL with T cell specific activators coated on a plate. The activators are antibodies specific to surface molecules whose activation correlates with interferon-gamma expression, namely, anti-CD3 (OKT3, 200 ng/ml Janssen-Cilag, Neuss, Germany), followed by phorbol 12-myristate 13-acetate (PMA) and ionomycin activation. TIL can then be stained with an anti-interferon-gamma antibody conjugated to phycoerythrin and captured on anti-PE microbeads run through a magnetic separator according to (Becker et al., 2001, Nature Med., 7:1159).

It is to be understood that variation will be found in different isolations of TIL from different patients, and thus, also taking into account variations in protocols, one of skill in the art will recognize that some experimentation may be necessary to determine the most effective amount of TIL for each individual patient in combination with Fas inhibitors. This additional experimentation is no more than routine and readily determined by the practitioner.

In Vivo Activation of Immune Cells

In yet another embodiment, the invention contemplates methods comprising co-administration of Fas inhibitors with an effective amount of immune cell activators. In this embodiment, the method increases the quantity of anti-tumor immune cells and/or activates the patient's anti-tumor immune response (e.g., via dendritic cells or cytotoxic T cells). In a specific example, Fas inhibitors can be used in combination with flt3-L to boost the patient's immune cell-mediated response, namely, dendritic cells, to tumor antigens (Pawlowska et al., 2001, Blood, 97:1474).

Further, Fas inhibitors can be used in combination therapies with one or more additional agents to enhance an immune response against cancer antigens. For example, CD40 binding proteins, which enhance the ability of dendritic cells to process and present antigens to effector T cells, can be administered in combination with Fas inhibitors to enhance an immune response. This method can also include the co-administration of additional factors in the treatment, including but not limited to flt3-L. Such immune responses can include responses against cancer antigens. Representative CD40 binding proteins useful in combination therapy with Fas inhibitors include CD40L and antibodies immunoreactive with CD40 which are described in U.S. Pat. No. 6,087,329 and PCT International Publications WO 93/08207 and WO 96/40918.

Additionally, 4-1BB-L and antibodies reactive with 4-1BB, both of which are T-cell co-activation factors, can be administered in combination with Fas inhibitors to enhance immune responses to cancer. 4-1BB-L and antibodies reactive with 4-1BB can be used in combination therapies to enhance immune responses to cancer antigens. 4-1BB-L and antibodies reactive with 4-1BB are described in U.S. Pat. No. 5,674,704.

Additionally, flt3-L, interferon alpha, RANKL, or a CD30 ligand antagonist can be administered in combination with Fas inhibitors to enhance immune responses. Other molecules that can be used in combination with Fas inhibitors according to the present invention include flt3-L, IL-2, IL-12, IL-15, TRAIL, VEGF antagonists, Tek antagonists, molecules that enhance dendritic cell function, survival, or expansion, molecules that enhance T cell activation or differentiation, molecules that enhance dendritic cell migration including various chemokines, molecules that increase the availability of target cell antigens, such as apoptotic factors and molecules that enhance MHC Class I presentation including the various interferon's, angiogenesis inhibitors, inhibitors of immunosuppressive molecules released by tumors including IL-10, VEGF, and TGF-β, and tumor-specific antibodies including toxin- or radio-labeled antibodies.

Pharmaceutical Preparations and Dosage

Compounds that antagonize Fas activity can be administered to a patient at therapeutically effective amounts to treat or ameliorate bone marrow failure or cancer. A therapeutically effective amount refers to that amount of the compound sufficient to result in amelioration of symptoms of, for example, bone marrow failure or cancer. Symptoms of bone marrow failure include fatigue, malaise (vague feeling of physical discomfort or uneasiness) sensitivity to cold, shortness of breath, dizziness and restless legs syndrome (uncomfortable feeling in legs, sensations of pulling, tingling, crawling, accompanied by a need to move the legs). Symptoms of cancer include pain, wasting and/or loss of appetite, tumor burden, nausea, fatigue, diarrhea, vomiting, and constipation.

When a Fas inhibitor is co-administered with another therapeutic agent, doses are modified according to any interactions that can occur between the therapeutic agents. An example of administration of dendritic cells is given in U.S. Pat. No. 5,788,963, to which the methods of the present invention are particularly well suited. In one embodiment, when lymphocytes are co-administered with a Fas inhibitor, the number of administered lymphocytes exceed the estimated number of cancer cells. For example, with LAK cells, 10 to 10 fold excess of LAK cells be administered relative to the cancer cells. In a more particular example that is not meant to be limiting, if a 1 cm tumor contains roughly 10⁸ tumor cells, then 10⁹ to 10¹⁰ LAK cells should be administered (Kimura and Yamaguchi, 1997, Cancer, 80:42-49).

It is to be understood that particular lymphocytes, such as TIL, may be more effective at clearing tumor cells than LAK cells and as such a lower dose may be administered according to the judgment of one of skill in the art. In addition, it is understood that lymphocytes may be more effective at clearing one type of cancer cell relative to another and as such, the dose of cells for administration can be adjusted accordingly (Hoffman et a., 2000, Seminars in Oncology, 27:221-233). The relative effectiveness of a lymphocyte for killing a cancer cell can be tested in various assays well known in the art, such as for example chromium release assays.

Toxicity and therapeutic efficacy of compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. A large therapeutic indices is indicative of higher therapeutic value in a clinical setting.

While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds can lie within a range of circulating concentrations that include the ED50 with little or no toxicity. The dosage can vary within this range depending upon the dosage form employed and the route of administration utilized. For any compound used in the method of the invention, the therapeutically effective amount can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture prior to administration to a patient.

When another therapeutic is administered in combination with the Fas antagonists (co-administration), the Fas antagonists can be delivered either prior to, simultaneous with, or after delivery of the second therapeutic. Simultaneous administration can encompass mixing the Fas antagonists with the second therapeutic prior to administration to the patient, or administration to the patient in separate infusions, albeit at the same time. It is also contemplated that the dose of the second therapeutic should be consistent with established therapeutic ranges, however, should there can be an increase in effectiveness of the therapeutic when used in combination with a Fas antagonist that is greater than the sum of either alone there is synergy. Thus, in the case of synergy, it will be understood that doses can be decreased relative to recommended ranges in light of enhanced effectiveness.

In one embodiment of the invention, a Fas antagonists is administered one time per week to treat the various medical disorders disclosed herein, in another embodiment is administered at least two times per week, and in another embodiment is administered at least once per day. An adult patient is a person who is 18 years of age or older. If injected, the effective amount, per adult dose, of a polypeptide inhibitor of Fas ranges from about 1-500 mg/m², or from about 1-200 mg/m², or from about 1-40 mg/m² or about 5-25 mg/m². Alternatively, a flat dose can be administered, whose amount can range from 2-500 mg/dose, 2-100 mg/dose or from about 10-80 mg/dose. If the dose is to be administered more than one time per week, an exemplary dose range is the same as the foregoing described dose ranges or lower. Such Fas antagonists can be administered two or more times per week at a per dose range of 25-100 mg/dose.

In one embodiment of the invention, the various indications described below are treated by administering a preparation acceptable for injection containing a Fas and/or FasL binding protein at 80-100 mg/dose, or alternatively, containing 80 mg per dose. If the Fas antagonist is an antibody, the dose can be from 0.1 to 20 mg/kg, and can be given intravenously as a 15-minute to 3-hour infusion. The dose is administered repeatedly at biweekly, weekly, or separated by several weeks.

If a route of administration of Fas signaling antagonist other than injection is used, the dose is appropriately adjusted in accord with standard medical practices. For example, if the route of administration is inhalation, dosing can be one to seven times per week at dose ranges from 10 mg/dose to 50 mg per dose. In many instances, an improvement in a patient's condition will be obtained by injecting a dose of up to about 100 mg of a soluble Fas inhibitor or FasL binding protein or an antagonistic antibody one to three times per week over a period of at least three weeks, though treatment for longer periods can be necessary to induce the desired degree of improvement. For incurable chronic conditions, for example, patients with bone marrow failure caused by a genetic disorder, the regimen can be continued indefinitely.

For pediatric patients (ages 4-17), a suitable regimen involves the subcutaneous injection of 0.4 mg/kg to 5 mg/kg of a Fas inhibitor such as a Fas and/or FasL binding protein, administered by subcutaneous injection one or more times per week.

Formulations and Use

Pharmaceutical compositions for use in accordance with the present invention can be formulated in conventional manner using one or more physiologically acceptable carriers or excipients. Thus, the compounds and their physiologically acceptable salts and solvates can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. The preparations can also contain buffer salts, flavoring, coloring and sweetening agents as appropriate. In addition, preparations for oral administration can be suitably formulated to give controlled release of the active compound.

For buccal administration the compositions can take the form of tablets or lozenges formulated in conventional manner.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

When the active ingredient is a protein such as a antibody or soluble extracellular domain of a ligand or receptor, the aqueous formulation will preferably also comprise a buffer, e.g., acetate, phosphate or histidine and be in the pH range of 4.0 to 7.2, or more preferably 4.8 to 5.6, a polyol, e.g., sorbitol, sucrose, or mannitol, and optionally a surfactant, e.g., polysorbate, and a preservative.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

Equivalents and References

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method for treating bone marrow failure comprising administering to a patient in need thereof an effective amount of an inhibitor of Fas mediated apoptosis.

2. The method of claim 1 wherein the inhibitor is a soluble extracellular domain of Fas.

3. The method of claim 2 wherein the soluble extracellular domain of Fas is fused to the Fc domain of an immunoglobulin molecule.

4. The method of claim 1 wherein the inhibitor is an antibody capable of inhibiting Fas mediated signaling.

5. The method of claim 4 wherein the inhibitor antibody is specific for Fas.

6. The method of claim 4 wherein the inhibitor antibody is specific for the Fas ligand.

7. The method of claim 1 further comprising co-administering an effective amount of a therapeutic selected from the group consisting of TNF inhibitors and antithymocyte globulin, and a growth factor.

8. The method of claim 7 wherein the TNF inhibitor is a soluble extracellular domain of the TNF-receptor fused to the Fc domain of an immunoglobulin.

9. The method of claim 7 wherein the growth factor is selected from the group consisting of TNF inhibitors, antithymocyte globulin, sargramostim, filgrastim, darbepoetin alfa, and erythropoietin.

10. The method of claim 1 wherein the bone marrow failure is selected from the group consisting of aplastic anemia, refractory anemia, and myelodysplastic syndrome.

11. A method of treating cancer comprising co-administering to a patient in need thereof an effective amount of an inhibitor of Fas mediated apoptosis and an immune cell therapy.

12. The method of claim 11 wherein the inhibitor is a soluble extracellular domain of Fas.

13. The method of claim 11 wherein the inhibitor is an antibody capable of blocking Fas mediated signaling.

14. The method of claim 13 wherein the inhibitor antibody is specific for Fas.

15. The method of claim 13 wherein the inhibitor antibody is specific for the Fas ligand.

16. The method of claim 11 wherein the immune cell therapy comprises antigen primed dendritic cells.

17. The method of claim 11 wherein the immune cell therapy comprises an effective amount of immune cells selected from the group consisting of lymphocyte activated killer cells and tumor infiltrating lymphocytes.

18. The method of claim 11 wherein the immune cell therapy comprises an effective amount of an immune cell activator selected from the group consisting of flt3-ligand, agonist binding proteins of CD40 including agonist antibodies to CD40 and CD40L or fragments of CD40L, 4-1BB-L, agonist antibodies to 4-1BB, 4-1BB-L, interferon alpha, RANKL, a CD30 ligand antagonist, GM-CSF, TNF-α, IL-3, IL-4, c-kit-ligand, and/or GM-CSF/IL-3 fusion proteins.

19. The method of claim 11 wherein the cancer is selected from the group consisting of autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemia, T gamma lymphoproliferative disease, cutaneous B cell lymphoma, cutaneous T cell lymphoma (i.e., mycosis fungoides), S+E, ezary syndrome, acute myelogenous leukemia, chronic or acute lymphoblastic leukemia, hairy cell leukemia, sarcoma, osteosarcoma, and carcinoma, such as adenocarcinoma, breast cancer, squamous cell carcinoma, Epstein-Barr virus-positive nasopharyngeal carcinoma, glioma, colon, stomach, prostate, renal cell, cervical and ovarian cancers, lung cancer (SCLC and NSCLC).
